(19) United States
(12) Patent Application Publication
(10) Pub. No.: US 2018/0303890 A1
(43) Pub. Date: Oct. 25, 2018

(54) A METHOD FOR MANUFACTURING SPECIAL EXTRACTS FROM SYMPHYTUM

(71) Applicant: Merck Patent GmbH, Darmstadt (DE)

(72) Inventor: Peter Häcker, Arbon (CH)

(21) Appl. No.: 15/768,892

(22) PCT Filed: Oct. 24, 2016
(86) PCT No.: PCT/EP2016/075487

§ 371 Date: Apr. 17, 2018

(30) Foreign Application Priority Data
Oct. 22, 2015 (EP) 15191070.0

(51) Int. Cl. A61K 36/30

(52) U.S. Cl. CPC A61K 36/30; A61K 2236/333; A61K 2236/55; A61K 2236/39; A61K 2236/15

(57) ABSTRACT

A method for producing a special extract from symphytum includes the steps of: preparing a suspension system from symphytum; differential precipitation of mucopolysaccharides in the suspension system, wherein the differential precipitation includes the following steps: first differential mucous precipitation of high molecular weight mucopolysaccharides as a first deposit in the suspension system, separating of the first deposit and an extraction residue as raffinate and obtaining of a first extract phase; second differential mucous precipitation of low molecular weight mucopolysaccharides as a second deposit from the first extract phase, separating of the second deposit and obtaining of a second extract phase; selective removal of pyrrolizidine alkaloids from the second deposit and/or the second extract phase; and combining of the second deposit, in particular the second deposit cleansed of pyrrolizidine alkaloids, and the second extract phase, in particular the second extract phase cleansed of pyrrolizidine alkaloids, to form the special extract.

### BACKGROUND OF THE INVENTION

### Field of the Invention

The invention relates to the field of manufacture of special extracts. In particular, it relates to a method for manufacturing a special extract from symphytum, to a special extract from symphytum as well as to a method for the manufacture of a first, mucilage-free and/or a second mucilaginous extraction phase, and to a method for precipitating mucopolysaccharides in a suspension system from symphytum as well as to a method for the selective removal of pyrrolizidine alkaloids from an extract phase from symphytum and/or from a precipitate from symphytum, said precipitate being separated from an extract phase.

### Description of Related Art

Symphytum, also called comfrey, is a traditional medicinal plant whose effect has already been known from ancient times. Anti-inflammatory, analgesic and tissue-regenerating properties are attributed to the comfrey plant, consisting of the fresh and/or dried, aboveground parts, as well as to the comfrey root, consisting of fresh and/or dried belowground parts. In the recent past, the indication range of comfrey root extracts has been confirmed by the proof of percutaneous effectiveness in the case of ankle distortions. Herein, comfrey has been found to be clinically effective and significantly superior with regard to the reduction of pain, inflammation, swelling, movement restriction and global effectiveness. Furthermore, modern test methods and application observations have confirmed the effectiveness and harmlessness in the case of various muscle and joint ailments. The medical application of preparations from all, in particular the belowground parts of the plant (symphytum) have thus become well established, and the preparations, apart from the treatment of bruises, strains or sprains, are also applied in the case of painful joint arthrosis and acute back pain.

The constituents of the comfrey plant contain 0.6-4.7% allantoin, a substantial amount of mucopolysaccharides (25-30%) including fructose units and glucose units, phenolic acids such as rosmarinic acid (up to 0.2%), chlorogenic acid (0.012%) as well as caffeic acid (0.004%) and alpha-hydroxy-caffeic acid, glycopeptides, amino acids and triterpensaponines in the form of monodesmosidic and bidesmosidic glycosides.

Allantoin, a purine derivative which is related to uric acid, mucopolysaccharides, tannins and triterpensaponines are considered as active ingredients, thus clinically effective constituents of symphytum. Rosmarinic acid and other hydroxycinnamic acid derivatives are of central importance for the pharmacodynamics. Probably all active ingredient groups are of relevance, but the active mechanisms however not completely understood.

All plant parts of symphytum likewise contain pyrrolizidine alkaloids in the form of the N-oxides. Symphytum includes roughly 150 different pyrrolizidine alkaloids, the most important being 7-acetyllycopsamine, 7-acetylintermedine together with small quantities of intermedine, lycopsamine and symphytine. The total quantity of pyrrolizidine alkaloids varies from 0.013% to 1.2% also for method-specific reasons (analysis). The pyrrolizidine alkaloids echimidine and symlandine are not found in Symphytum officinale. Concerning the pyrrolizidine alkaloids, it is possibly the case of mutagenic or carcinogenic substances, which is why the internal application of the drug, as well as of preparations which are manufactured therefrom, has been abstained from. However, the very popular external application given intact skin appears to be very justifiable, which is why in Europe, on the part of authorities, defined limit values for daily doses of maximal 0.35 micrograms of pyrrolizidine alkaloids are tolerated. At all events, it is desirable to keep the content of pyrrolizidine alkaloids as low as possible from a toxicological point of view and for precautionary reasons.

What is known from the industrial field is a method that is based on an alcoholic extract from Petasites hybridus being treated with a cation exchanger for depleting the pyrrolizidine alkaloids. More than 93% of the pyrrolizidine alkaloids can be removed from these extracts in this manner (see Ch. Mauz et. al. in Pharmaceutica Acta Helvetiae, Volume 60 (1985), 256-259). In contrast to symphytum, petasites has no mucopolysaccharides.

The huge active potential of the comfrey plant, which has been recognised by suitable studies, justifies the optimisation of a technological-methodical extract formation, which is why it is therefore the object of the present invention at least to provide a method for the manufacture of special extracts from symphytum, the method permitting a high yield of pharmaceutically relevant ingredients by way of method technology that is essentially gentle on the product.

### SUMMARY OF THE INVENTION

It is therefore the object of the invention, to provide a method for manufacturing a special extract from symphytum, the special extract from symphytum as well as a method for manufacture a first, mucilage-free and/or a second mucilaginous extraction phase, and a method for precipitating mucopolysaccharides in a suspension system from symphytum, as well as a method for the selective removal of pyrrolizidine alkaloids from an extract phase and/or from a precipitate which is separated from an extraction phase, which permit an optimised manufacture of the special extract.

This object or an essential part of this object is achieved by a method for manufacturing a special extract from symphytum, as well as a method for manufacturing a first, mucilage-free extraction phase from symphytum, a method for manufacturing a second, mucilaginous extraction phase from a first mucilage-free extraction phase, a method for precipitating mucopolysaccharides in a mucilaginous suspension system from symphytum, a method for the selective removal of pyrrolizidine alkaloids from a precipitated precipitate from symphytum, said precipitate being separated from an extract phase, a method for the selective removal of pyrrolizidine alkaloids from an extract phase from symphytum, and a special extract from symphytum, with the features of the respective independent patent claims.

The method for manufacturing a special extract from symphytum includes at least one, in particular all, of the following steps:
providing a suspension system from symphytum,
differential precipitation of mucopolysaccharides in the suspension system, wherein the differential precipitation includes the following steps:
   first differential mucilage precipitation of high-molecular mucopolysaccharides as a first precipitate in the suspension system, separating the first precipitate and an extraction residue as a raffinate and obtaining a first extract phase;
   second differential mucilage precipitation of low-molecular mucopolysaccharides as a second precipitate from the first extract phase, separating the second precipitate and obtaining a second extract phase;
selective removal of pyrrolizidine alkaloids from the second precipitate and/or the second extract phase; and
pooling the second precipitate, in particular the second precipitate, which is purified of the pyrrolizidine alkaloids, and the second extract phase, in particular the second extract phase, which is purified of the pyrrolizidine alkaloids, into the special extract.

A suspension system from symphytum can be mucilaginous and viscous on account of this, since symphytum includes mucilaginous substances, so-called mucopolysaccharides. Viscous in this context means more viscous than water at room temperature. The mucopolysaccharides dissolve colloidally in the suspension system or as a constituent of extractive substances are lysed or swelled in the suspension system. In other embodiments, it is possible to provide an extract solution from symphytum instead of a suspension system. Such an extract solution could be provided by way of macerating plant parts in a solvent, in particular an alcoholic solvent, in particular an aqueous-alcoholic solvent. On manufacturing such an extract solution, the extraction solution is not necessarily present in suspended form, and for this reason the subsequent description of the invention can also be conferred upon extraction systems which are manufactured according to other extraction methods, instead of the suspension system. In such a case, the reading of the description must be adapted accordingly.

It is to be noted that symphytum is a generic term for all symphytum sub-species. Symphytum can include for example Symphytum officinale L. Not only can the methods described here be applied to a special sub-species of symphytum, such as for example Symphytum officinale L., but also to all symphytum species. That which is preferably used for the method is Symphytum officinale, Symphytum x uplandicum, Symphytum asperum, Symphytum bulbosum, Symphytum tuberosum and/or Symphytum peregrinum, but is its Symphytum officinale, which is particularly preferred.

Special extracts, for example from plants, differ from conventional extracts in that on manufacture, yet further processing steps are carried out apart from the pure extraction. With regard to the manufacture of a special extract, it is the case of a complex, multi-staged extraction and purification process. Herein, undesired ingredients, such as for example pyrrolizidine alkaloids, are removed and the desired active ingredients, which determine the effectiveness of the special extract, are concentrated or enriched.

The extraction residue is to be understood as the solid residual phase or raffinate and it includes for example the solid plant parts, which are not dissolved in the extract phase.

In other embodiments, it is possible for the extraction residue to be separated from the suspension system before the precipitation of the mucopolysaccharides. In this case, with the first differential mucilage precipitation, the first precipitate is separated from the first extract phase, since the extraction residue has already been separated as a raffinate from the suspension system.

The use of special extracts has several advantages. The active ingredient concentration in the special extract can thus be increased. Undesirable by-products are removed on extraction or further processing steps and the special extract becomes more tolerable by way of this. Furthermore, the composition and the quantity of ingredients can possibly be standardised and/or quantified. This ensures a constant quality of the special extract.

With regard to the method for manufacturing a special extract from symphytum, high-molecular mucopolysaccharides can be precipitated as a precipitate in the mucilaginous suspension system and be separated from an extraction phase (fluid phase) after the precipitation. The extract phase can also be called a primary extract. The precipitate and the separated primary extract or the separated extract phase can herein be individually purified of plant substances with a harmful potential, such as for example pyrrolizidine alkaloids, and can be unified or brought together again after the purification of pyrrolizidine alkaloid. By way of this, it is possible for the special extract to only have a low pyrrolizidine alkaloid content, by which means the application of the special extract in the therapeutic field can be rendered possible.

The precipitation of colloidally dissolved mucopolysaccharides in the suspension system from symphytum as a precipitate can include a differential mucilage precipitation. Differentially or stepwise specific extractive substances such as undesired and/or effective mucopolysaccharides can be precipitated out of the suspension system in this manner, and possibly be separated from the extract phase as insoluble aggregates.

The special extract can include a pyrrolizidine alkaloid content of maximally 0.5 ppm, in particular maximally 0.1 ppm. By way of this, it is possible for the special extract from symphytum to be further processed into a cream and/or ointment without any misgivings, and such a cream or ointment can be used for therapeutic treatment, in particular for the therapeutic treatment of pain, inflammation, swelling, movement restrictions, bruises, strains, sprains, joint arthrosis, back pain, muscle ailments and/or joint ailments.

The suspension system, in which the mucopolysaccharides are colloidally dissolved can be manufactured and/or provided by way of a discontinuous extraction. By way of this, it is possible to effectively extract different extractive substances from the plant symphytum during the individual discontinuous extraction steps.

Herein, the discontinuous extraction can include a polarity-controlled discontinuous extraction, wherein the discontinuous extraction can be achieved or rendered possible by way of the multi-stage setting of the polarity of the extraction phase. The discontinuous or also multi-stage extraction in the present case can also be indicated as a fractional extraction.

The discontinuous extraction can include the following steps:
providing a suspended, first, essentially mucilage-free extraction phase from symphytum; and
manufacturing a second, mucilaginous extraction phase.

In the first extraction phase, the mucopolysaccharides are not yet colloidally dissolved, thus not yet lysed or swelled. For this reason, the first extraction phase is low-viscous and is uninfluenced by the mucopolysaccharides. On account of this, it is possible for the extractive substances, such as for example the active ingredients, to be efficiently released and simply dissolved in the essentially mucilage-free, first extraction phase. The presence of mucilaginous substances, such as for example mucopolysaccharides renders the diffusion processes more difficult and therefore the extraction of active ingredients from symphytum. Furthermore, the mucopolysaccharides can be colloidally dissolved and lysed in a second step on manufacturing the mucilaginous second extraction phase from symphytum and can therefore be rendered accessible to the special extract in a partially suitable and simple manner.

The first, essentially mucilage-free extraction phase can be manufactured or provided by a method, including at least one of the following steps:
providing fresh, frozen and/or dried plant parts of symphytum;
adding low-molecular alcohol, in particular ethanol, to the plant parts;
setting the alcohol content of the first extraction phase (1) to 65-75% by volume, in particular to 70% by volume.

Low-molecular alcohol is to be understood as an alcohol which includes maximally ten C-atoms, for example ethanol, propanol, isopropanol etc. A preferred alcohol is ethanol.

The method for manufacturing an essentially mucilage-free first extraction phase as such and the method for manufacturing a special extract share a common inventive idea, specifically the improvement or simplification of the manufacture of a special extract from symphytum. For this reason, the method for manufacturing the first, essentially mucilage-free extraction phase from symphytum can also be considered as an independent method or in combination with the method for manufacturing the special extract or individually in combination with one of the other methods, which are described in this text.

The provided fresh, frozen and/or dried plant parts of symphytum can include aboveground and/or belowground plant parts of symphytum. Herein, the frozen plant parts can also be deep-frozen. The use of deep-frozen plant parts has the advantage that enzymatic decomposition processes in the plant parts, for example after the harvest, can be prevented or minimised. Biochemical processes of post-harvest physiology and deterioration can be prevented by way of this, which therefore ensures an optimal conservation of any active ingredients.

In embodiments of the invention, the plant parts are provided in a fresh, frozen, deep-frozen and/or dried form, pre-comminuted into a size of 0.5 cm to 2.5 cm.

In embodiments of the invention, the pre-comminuted plant parts are further comminuted by way of wet-comminution and/or turbo-extraction, wherein the plant parts can be comminuted to a size of 0.1 mm to 1 mm. A suspension system of the finely comminuted plant parts and of a solvent which is added to the pre-comminuted plant parts for the wet-comminution and/or the turbo-extraction arises in this manner. The solvent can thereby include low-molecular alcohol with an alcohol content of 65-75% by volume, in particular of 70% by volume. Concerning the wet comminution and/or turbo-extraction, a comminution of the starting material (pre-comminuted plant parts) occurs due to the cutting forces and shearing forces, and the cell walls are thereby torn open several times. Herein, the cell walls of the plant material are broken open in an optimal manner. Apart from a comminution of the plant particles, a suspension occurs with the help of the shear forces. In other words: on wet-comminution and/or turbo-extraction, the plant parts are greatly comminuted amid the application of a solvent, by which means the surface area of the plant parts is greatly increased and a cell lysis thus rendered possible and the contact between the solvent and the plant parts is optimised for the extraction. Apart from an additional comminution, the diffusion and dissolving procedures are greatly accelerated. The turbo-extraction can also be indicated as a vortex extraction.

On wet-comminution, the pre-comminuted plant parts are comminuted to the desired size in a suitable cutting and/or grinding device amid the addition of a low-molecular alcohol. The low-molecular alcohol can herein include an alcohol content of 65-95% by volume, in particular 65-70% by volume, in particular 70% by volume.

In contrast to grinding and/or comminution of the plant parts in air, the oxidation of the plant parts can be reduced by way of the wet-comminution and/or turbo-extraction of the fresh and/or frozen and/or dried plant parts of symphytum amid the addition of alcohol and/or an arbitrary other suitable solvent. By way of this, it is not only possible for an oxidative conversion of the active ingredients of the symphytum to be able to be reduced or largely prevented, but also enzymatic reactions in the extraction phase.

A swelling, dissolving and/or lysis of the mucopolysaccharides is prevented by way of setting the alcohol content to 65-95% by volume, by which means it is mainly mucilage-free active ingredients which are dissolved out of the plant parts. For example, polyphenols which make up a part of the active ingredients of symphytum can be dissolved particularly well in the extraction phase, from an alcohol content of above 65% by volume. The extraction for example of polyphenols can be effected particularly efficiently due to the fact that the first, essentially mucilage-free extraction phase only includes a minimal content of mucopolysaccharides.

In particular, the alcohol content of the first extraction phase can be set to 70% by volume. It is particularly with such an alcohol content, in particular in combination with an increased temperature of 45° C.80° C., than an anti-bacterial effect of the first extraction phase can be ascertained. In this manner, it is possible for the first, essentially mucilage-free extraction phase to develop a germ-reducing effect, which amongst other things can also be advantageous for the further processing and/or for the use in a special extract, as well as can ensure the adherence to legal requirements.

On manufacture of the first extraction phase, the temperature of the extraction phase can be increased to 45° C. to 80° C., in particular to 50° C.-70° C., in particular to 54-56° C. By way of increasing the temperature, on the one hand the exchange processes can be improved on exaction of the active ingredients and on the other hand possible enzymes and/or proteins which are present in the extraction phase can be (selectively) denatured. Enzymatic degradation and/or conversion processes in the mucilage-free, first extraction phase can be prevented by way of this.

The mucilaginous, second extraction phase can be manufactured by way of maceration, in particular by way of stirring maceration of the first extraction phase, wherein the alcohol content (low-molecular alcohol) of the second extraction phase is set to 45% by volume-55% by volume, in particular to 49% by volume to 51% by volume, in particular to 50% by volume.

The method for manufacturing a mucilaginous, second extraction phase as such and the method for manufacturing a special extract share an inventive idea, specifically the improvement of the manufacture of a special extract from symphytum by way of optimised method steps or part-steps. For this reason, the method for manufacturing the second, mucilaginous extraction phase from symphytum can also be considered as an independent method or in combination with the method for manufacturing the special extract.

The setting of the alcohol content can be effected by way of the addition of water to the first, essential mucilage-free extraction phase. The polarity of the extraction phase is increased by way of this, which leads to the swelling and herewith to a colloidal dissolving of the mucopolysaccharides. In this manner, it is possible for the second extraction phase or the suspension system from symphytum to have a high share of active ingredients. A further step of the discontinuous extraction can be achieved by this.

The water content of the extraction phase can be increased and the alcohol content of the mucilaginous, second extraction phase can be set to 45% by volume-55% by volume, amid constant stirring. The polarity of the second extraction phase is increased in comparison to the first extraction phase in this manner. By way of this, it is possible for the mucilaginous substances, such as for example the mucopolysaccharides, to swell and be colloidally dissolved out of the plant parts of symphytum and extracted. Consequently, a suspension system, which corresponds to the second mucilaginous extraction phase is created.

For this reason, the second extraction phase, apart from the colloidally dissolved, lysed mucopolysaccharides, also includes a high concentration of active ingredients, for example the polyphenols, which are highly soluble in the first extraction phase. The second extraction phase can also be called a suspension system, which is mucilaginous.

The mucilaginous, second extraction phase can be manufactured at room temperature. However, the manufacture of the second extract phase is also possible at higher temperatures, as long as a decomposition of thermolabile mucopolysaccharides and/or the conversion of the mucopolysaccharides are not promoted.

The method for precipitation, in particular for the differential precipitation, of mucopolysaccharides in a suspension system from symphytum, as a precipitate, as such shares an inventive idea with the method for manufacturing a special extract, specifically the improvement or simplification of the manufacture of a special extract from symphytum. For this reason, the method for the differential precipitation of mucopolysaccharides in the suspension system as a precipitate can also be considered as an independent method or in combination with the method for manufacturing the special extract or individually in combination with one of the other methods which are described in this text. Herein, mucilaginous suspension systems from symphytum which are manufactured or provided by way of an alternative extraction and not with the help of a discontinuous extraction can also be used.

As already described above: the precipitation of mucopolysaccharides as a precipitate can include a differential mucilage precipitation. Specific substances, such as undesired and/or effective mucilaginous substances can be precipitated differentially in the suspension system and possibly separated from the extract in this manner.

The differential mucilage precipitation can include at least one of the following steps:
first differential mucilage precipitation of high-molecular mucopolysaccharides as a first precipitate in a provided suspension system, separating a first precipitate, possibly as part of an extraction residue and obtaining a first extract phase;
second differential mucilage precipitation of low-molecular mucopolysaccharides as a second precipitate in the first extract phase, as well as separating the second precipitate and obtaining a second extract phase;
in particular drying the second precipitate after the separation.

In this manner, it is possible for the high-molecular mucopolysaccharides to be able to be separated from the mucilaginous suspension system by way of the first differential mucilage precipitation. The separation can be effected by way of sedimentation and subsequent siphoning of the first precipitate, possibly as part of an extraction residue, and in particular by way of pressing out. However, the separation can also be effected by way of other known methods, such as for example filtration, decanting, sieving, centrifuging and/or pressing

The raffinate, which apart from the first precipitate can also include the extraction residue, can be recycled for the method for manufacturing the special extract. It becomes possible for the high-molecular mucopolysaccharides of the first precipitate to possibly be put to another use by way of this.

The first extract phase can be specifically separated from the high-molecular mucopolysaccharides with the help of the first differential mucilage precipitation. In contrast to the low-molecular mucopolysaccharides, the high-molecular mucopolysaccharides do not penetrate the skin and therefore cannot presently be used as a potential active ingredient component. The high-molecular mucopolysaccharides have no therapeutic potential from today's perspective. Due to this, on the one hand the ineffective high-molecular mucopolysaccharides are removed from the extract and on the other hand the first extract phase becomes thinner than the suspension system and thus can be processed better.

With the second differential mucilage precipitation, the low-molecular mucopolysaccharides which are of interest as active ingredients for the special extract can be specifically precipitated out of the thin first extract phase after the separation of the high-molecular mucopolysaccharides and, as a disperse, second precipitate, be separated from the fluid, second extract phase. The second precipitate can be separated and thus obtained from the second extract phase by a known method, for example filtering, decanting, sieving, centrifuging and/or pressing. A second colloidally disperse precipitate, which includes the low-molecular mucopolysaccharides and which is mostly present in colloidally dissolved form in the second extract phase, arises by way of this. In dried form, the second precipitate can be present as a very fine-powdered precipitate.

The second precipitate can be dried after the separation. It is possible for the precipitate to be stored in the dried condition on account of this. The second precipitate can for example be lyophilised. Since lyophilisation is a particularly gentle drying method that is carried out at a low pressure, an oxidation of the second precipitate can be prevented. However, any other drying methods for drying the second precipitate are conceivable.

Concerning the first differential mucilage precipitation, the pH-value of the mucilaginous suspension system from symphytum can be adjusted to 4 to 5, wherein the suspension system can have a pH value of 6 to 6.5 before the precipitation. The precipitated-out, high-molecular mucopolysaccharides can be separated away, as described beforehand. After separation of the first precipitate from the first extract phase, the first extract phase has a lower viscosity than the mucilaginous suspension system. The first extract phase can be further processed to a better extent on account of this, and the ability to be handed for further washing and/or precipitation processes is improved.

It is to be noted that the polarity of the extraction phase is changed during the first differential mucilage precipitation by way of adjusting the pH value to 4-5. An additional extraction of active ingredients from the suspended plant parts, which are present in the suspension system and therefore an increase on the active ingredient concentration in the first extract phase, can be rendered possible by way of this. In other words: the multi-stage, discontinuous extraction can be optimised by a further method step or by a further pH-modified stage, by way of the additional extraction which can occur with the first differential mucilage precipitation and the change of the polarity which this entails. In the case that the suspension system from symphytum is provided by way of the described discontinuous extraction, the first extraction phase is manufactured in a first step. As described, alcohol-soluble extractive substances are specifically dissolved out of the plant parts in this first extraction phase with a relatively high alcohol content. The second extraction phase with a lower alcohol content is manufactured in a second step, and herein the mucopolysaccharides are lysed and colloidally dissolved by way of swelling. In a third step, extractive substances can further be dissolved out of the plant parts in an acidic environment on account of the renewed change of the polarity. The third step of course is only possible if the extraction residue has not been removed before the first differential mucilage precipitation.

Concerning the second differential mucilage precipitation of low-molecular mucopolysaccharides in the first extract phase, the pH value of the first extract phase can be set to 2.5-3.5. A fine, colloidally disperse second precipitate which can firstly remain in suspension is formed in this manner, by which means a good separation without the formation of lumps is rendered possible. The fine, second precipitate can cave a light-beige colour. The precipitate can be efficiently purified from undesired accompanying substances, such as for example pyrrolizidine alkaloids, due to the fine surface structure of the precipitated second precipitate. Furthermore, the fine, second precipitate can be separated from the first extract phase by way of the already described and known methods.

In contrast to this, a rubber-like, dark-brown agglomerate forms by way of a precipitation of the mucopolysaccharides in a single step, for example with ethanol. The agglomerate, in this form, renders impossible any removal of pyrrolizidine alkaloids.

The pH value of the mucilaginous suspension system, in particular of the second mucilaginous extraction phase and/or of the first extract phase can be set with the help of a catalyser and/or with the help of an acid. The catalyser, also called cation exchanger, can be an acidic catalyser based on a gel-like polymer. The catalyser can be simply removed from the extraction solution, for example by way of sieving, in this manner

The acidic catalyser can include sulphonic acid groups on the polymer surface. An ion exchange can take place on the surface of the catalyser in this manner, wherein the cations are bound. Mucopolysaccharides selectively precipitate out of the extract (suspension system and/or first extract phase) by way of the reduction of the pH value and form a (first and/or second) precipitate. Ionic pyrrolizidine alkaloids can additionally be accumulated on the catalyser due to ion exchange, by which means a part-reduction of undesired pyrrolizidine alkaloids in the extract, in particular in the first extract phase and/or in the second extraction phase occurs. In other words: the pH value of the suspension system and/or of the first extract phase can be set with the help of a cation exchanger in acidic form, wherein the cation exchanger can be designed on the basis of an organic polymer resin and wherein the cation exchanger can include in particular sulphonic acid groups on the polymer surface.

In further embodiments, the acids for adjusting the pH value can be a mineral acid or organic acid. Here too, a selective precipitation of the mucopolysaccharides occurs by way of a reduction of the pH value, as already described, but without a depletion of undesired pyrrolizidine alkaloids.

On using the acidic catalyser for adjusting the pH-value of the suspension system to 4-5 for the first, differential mucilage precipitation, the extraction solution can already be pre-purified of pyrrolizidine alkaloids, since the acidic catalyser can bind a part of the dissolved pyrrolizidine alkaloids on the catalyser surface.

The purification of the second precipitate by way of the selective removal of pyrrolizidine alkaloids from the second precipitate can include the following steps:
resuspending the second precipitate in a washing solution, in particular in a washing solution including an acidic, low-molecular alcohol,
separating the washed, second precipitate from the washing solution.

On resuspending the second precipitate in the washing solution, the mucopolysaccharides of the precipitate remain insoluble and the pyrrolizidine alkaloids are dissolved in the washing solution and can therefore be washed or extracted out of the precipitate. This can be rendered possible by way of the alcohol content of the washing solution being 65-95% by volume, in particular 78% by volume-82% by volume. The second precipitate, which is purified of the pyrrolizidine alkaloids after the washing, can be separated from the washing solution in a known way and manner by way of this, for example by way of sedimenting or siphoning or similar method steps. A washing solution with an alcohol content of 65-95% by volume is hereinafter also called a high-percentage alcohol.

The pH-value of the acidic washing solution can be 2 - 4, in particular 2.5 -3.5, in particular at least 3.

On resuspending, a ratio of precipitate to washing solution of 1:10 can be used. The ratio relates to the mass of the precipitate and of the washing solution.

The purification of the second precipitate by way of the selective removal of pyrrolizidine alkaloids from the second precipitate can alternatively include the following steps:
resuspending the second precipitate, in particular a lyophilised, second precipitate, in high-percentage, low-molecular alcohol, in particular ethanol,
adding a catalyser, in particular an acidic catalyser based on a gel-like polymer to the resuspended second precipitate, wherein the catalyser in particular includes sulphonic acid groups on the polymer surface, and
separating the catalyser from the resuspended, second precipitate and the subsequent recovery of the purified, second precipitate from the purified suspension.

It is possible to be able to purify the second precipitate of pyrrolizidine alkaloids by way of this. The mucopolysaccharides do not dissolve in high-percentage alcohol, but the pyrrolizidine alkaloids are highly soluble in alcohol. The pyrrolizidine alkaloids can be dissolved out of the second precipitate in this manner, without the effective mucopolysaccharides dissolving out of the second precipitate. As already described, the pyrrolizidine alkaloids can be bound on the catalyser out of the suspension by way of ion exchange by way of adding the acidic catalyser to the resuspended second precipitate, and be removed. The quantity of acidic catalyser can be adapted to the quantity of resuspended second precipitate, wherein in particular the same quantities of catalyser and precipitate can be used for the purification. In this context, the same quantity means the same mass. The catalyser can be separated from the fine, resuspended second precipitate and the second precipitate can subsequently be separated from the fluid, high-percentage alcohol solution. As already described, the separation can be effected by way of a known method, such as for example sedimenting and siphoning or decanting or the like.

The high-percentage, low-molecular alcohol can include 80% by volume to 98% by volume, in particular 96% by volume of low-molecular alcohol.

On resuspending the second precipitate, the quantity (mass) of the precipitate can be adapted to the quantity of the alcohol. Herein, a part of the second precipitate can be resuspended in 5 to 10 parts alcohol.

The resuspending and pyrrolizidine-alkaloid-purification can possibly be repeated with the purified second precipitate, by which means the pyrrolizidine alkaloid content of the precipitate can be further reduced.

The purified second precipitate can include maximally 1 ppm, in particular maximally 0.5 ppm, in particular maximally 0.1 ppm pyrrolizidine alkaloids. From a toxicological point of view, it is possible for the purified second precipitate to be able to be used in the special extract without misgivings by way of this.

The selective removal of pyrrolizidine alkaloids (PA) from the second extract phase can include at least one of the following steps:
pyrrolizidine-alkaloid-purification of the second extract phase with the help of a stationary phase, wherein the stationary phase in particular includes a cation exchanger in acidic form;
adjusting the pH value of the pyrrolizidine-alkaloid-purified second extract phase to 5.5-7, in particular to 6.2-6.5; and
concentrating the pyrrolizidine-alkaloid-purified second extract phase, in particular by way of distillation,
   wherein the purified, in particular concentrated, second extract phase, in particular the purified, second extract phase includes maximally 0.5 ppm pyrrolizidine alkaloids, in particular maximally 0.25 ppm pyrrolizidine alkaloids, in particular maximally 0.1 ppm pyrrolizidine alkaloids.

The pyrrolizidine-alkaloid-purification of the second extract phase with the help of a stationary phase can be realised for example by way of chromatographic, in particular column-chromatographic purification. Pyrrolizidine alkaloids can be removed from the second extract phase by way of the chromatographic, in particular column-chromatographic purification of the extract phase, in particular of the second extract phase. In particular, the removal of the pyrrolizidine alkaloids can be effected by way of the already described acidic catalyser on the basis of a gel-like polymer. Herein, the catalyser can include sulphonic acid groups on the polymer surface. An ion exchange can take place in this manner, said exchange able to be described by the following formula:

catalyser—H+PA⁺→catalyser—PA⁺

wherein catalyser —H is the acidic catalyser, PA⁺an ionic pyrrolizidine alkaloid, catalyser—PA a bound form of catalyser and pyrrolizidine alkaloid and H⁺a proton. By way of this, it is possible for the pyrrolizidine alkaloids to be bound on the catalyser and for the second extract phase to be purified from the pyrrolizidine alkaloids. Since the second extract phase has a relatively low viscosity after the separation of the precipitate, the second extract phase is particularly well suited to the chromatographic purification with a catalyser without a precipitation reaction.

The pH value of the second extract phase can be adjusted to 5.5-7, in particular to 6.2-6.5, after the purification of the second extract phase. The further processing, such as the concentrating, of the purified extract phase can be improved by way of this.

Furthermore, the purified second extract phase can be concentrated. The active ingredient concentration of the pyrrolizidine-alkaloid-purified second extract phase can be increased by way of the reduction of the volume entailed by this, wherein the substance quantity of active ingredients or extractive substances remains the same. The manufacture of the special extract can be improved in this manner and a drug-extract ratio (DER) can be defined. The concentrating can be carried out, for example, with the help of a distillation, in particular a vacuum distillation.

For example, the alcohol content of the special exact can be reduced after the mixing by way of the prior concentrating of the purified extract phase, in particular of the purified second extract phase. The stability of the special extract on further processing into a galenic preparation can be improved by way of this.

In an embodiment, the method for manufacturing a special extract from symphytum with a pyrrolizidine alkaloid content of maximally 0.5 ppm, in particular maximally 0.1 ppm can include the following steps:
providing a suspended, first, mucilage-free extraction phase from symphytum;
manufacturing a second, mucilaginous extraction phase;
first differential mucilage precipitation of high-molecular mucopolysaccharides as a first precipitate in a second, mucilaginous extraction phase, separating the first precipitate and an extraction residue as a raffinate and obtaining a first extract phase;
second differential mucilage precipitation of low-molecular mucopolysaccharides as a second precipitate from the first extract phase, separating the second precipitate and obtaining a second extract phase;
selective removal of pyrrolizidine alkaloids from the second precipitate;
selective removal of pyrrolizidine alkaloids from the second extract phase; and
pooling the pyrrolizidine-alkaloid-purified, second precipitate and the pyrrolizidine alkaloid-purified second extract phase, in particular a concentrated pyrrolizidine-alkaloid-purified second extract phase, into a special extract.

A method, with which a special extract with a pyrrolizidine alkaloid content of maximally 0.5 ppm, in particular maximally 0.25 ppm can be manufactured is provided in this manner.

At least one of the following steps and/or part steps:
providing a suspension system from symphytum;
first differential mucilage precipitation of high-molecular mucopolysaccharides;
second differential mucilage precipitation of low-molecular mucopolysaccharides;
selective removal of pyrrolizidine alkaloids from the second precipitate and/or from the second extract phase can be carried out under inert gas. It is possible to prevent or reduce an oxidation of the starting substances or of the intermediate products by way of this. Furthermore, undesired, enzymatic reactions can be minimised.

In a further aspect, the invention relates to a special extract, which can be manufactured with the described method.

The special extract from symphytum can be used in therapy.

The special extract from symphytum in particular can be used for external application in therapy.

It is also possible for the special extract from symphytum to be able to be applied for cosmetic purposes.

In particular, the special extract can be used in the therapy of pain, inflammation, swelling, movement restrictions, bruises, strains, sprains, joint arthrosis, back pain, muscle complains and/or joint complaints.

As already described, an aspect of the invention for improving the manufacture of a special extract from symphytum relates to a method for manufacturing an essentially mucilage-free, suspended first extraction phase from symphytum including the following steps:
providing fresh, frozen and/or dried plant parts of symphytum;
adding low-molecular alcohol, in particular ethanol, to the plant parts;
setting the alcohol content of the first extraction phase to 65-75% volume, in particular to 70% by volume.

A further aspect of the invention for improving the manufacture of a special extract from symphytum relates to a method for manufacturing a second, mucilaginous extraction phase from a provided first, essentially mucilage-free extraction phase from symphytum, wherein the second, mucilaginous extraction phase is manufactured by way of maceration, in particular by way of stirring maceration, of the first extraction phase, wherein the alcohol content of the extraction phase is set to 45% by volume 55% by volume, in particular to 50% by volume, as already described.

A further aspect of the invention for improving the manufacture of a special extract from symphytum relates to a method for precipitating mucopolysaccharides (MPS) including a differential mucilage precipitation, wherein the differential mucilage precipitation includes the following steps:
first differential mucilage precipitation of high-molecular mucopolysaccharides as a first precipitate in a suspension system from symphytum, separating the first precipitate and possibly an extraction residue as a raffinate and obtaining a first extract phase;
second differential mucilage precipitation of low-molecular mucopolysaccharides as a second precipitate from the first extract phase, separating the second precipitate and obtaining a second extract phase.

A further aspect of the invention for the improvement of a special extract from symphytum relates to a method for the selective removal of pyrrolizidine alkaloids (PA) from a precipitate, which is precipitated from a suspension system from symphytum and is separated from an extract, including the following steps:
resuspending the precipitated and separated precipitate in a washing solution, in particular in a washing solution including an acidic, low-molecular alcohol, wherein the alcohol in particular has a pH-value of 2-4, and
separating the washed precipitate from the washing solution; or
resuspending the precipitated and separated precipitate in high-percentage, low-molecular alcohol, in particular ethanol,
adding a catalyser, in particular an acidic catalyser on the basis of a gel-like polymer, to the resuspended precipitate, wherein the catalyser in particular includes sulphonic acid groups on the polymer surface, and
separating the catalyser from the resuspended precipitate and subsequent recovery of the purified precipitate from the suspension;
   wherein the resuspending and purifying with the purified precipitate is repeatable; and wherein the purified precipitate in particular includes maximally 1 ppm, in particular maximally 0.5 ppm, in particular maximally 0.1 ppm of pyrrolizidine alkaloids.

In a further aspect, the invention relates to the improvement of the manufacture of a special extract from symphytum for the selective removal of pyrrolizidine alkaloids from an extract phase, which is precipitated and separated from a precipitate in a suspension system from symphytum, including at least one of the following steps:
purifying the extract phase with the help of a stationary phase, wherein the stationary phase in particular includes a cation exchanger in acidic form;
adjusting the pH-value of the purified extract phase to 5.5-7, in particular to 6.2-6.5; and
concentrating the purified extract phase, in particular by way of distillation, in particular by way of vacuum distillation,
   wherein the purified, in particular concentrated extract phase includes maximally 0.5 ppm, pyrrolizidine alkaloids, in particular maximally 0.1 ppm pyrrolizidine alkaloids. Further preferred embodiments are to be derived from the dependent patent claims. Herein, features of the method claims, where appropriate can be combined with the method claims and vice versa. The independent methods can also be combined with one another in an arbitrary combination.

### EXAMPLE

Belowground parts of Symphytum officinale L in a cut size of 5 mm-10 mm in ethanol 70% by volume amid stirring with a turbo-mixer and at a continuously increasing temperature up to 55° C. are subjected to a wet-comminution into particle sizes of 0.1 mm-1 mm. A first, essentially mucilage-free extraction phase 1 is manufactured in this manner. A movement maceration is effected subsequently to this at room temperature by way of adding purified water for 180 minutes, by which means the second, mucilaginous extraction phase 2 is manufactured, wherein this has a higher polarity than the first, mucilage-free extraction phase 1.

A first, differential mucilage precipitation step is initiated by way of adding acidic catalyser in protonised (ft) form amid further stirring. The catalyser is an acidic catalyser based of a gel-like polymer and includes sulphonic acid groups on the polymer surface. Herein, the pH-value of the second extraction phase 2 is adjusted to 4-5 and high-molecular mucopolysaccharides are precipitated as a first precipitate N1. A separation of the fluid extract phase, also called extract phase A, from the disperse extraction residue is effected by way of pressing out the sediment phase or the extraction residue, after a further extraction step (pH-modified). The first extract phase A arises by way of this procedural method and as a filtrate is subjected to a further proton exchange amid the addition of the aforementioned acidic catalyser, for at least 60 minutes, until the stable reaching of pH 2.5-3.5. A quantitative precipitation of the mucopolysaccharides as a second precipitate N2 occurs with the reduction of the pH value and whilst stirring the first extract phase A. The acidic catalyser is subsequently sieved off and the disperse second precipitate N2, also called mucilage precipitate, is removed from the arisen second extract phase B by way of centrifuging the disperse system.

The second precipitate N2 resulting according to the preceding procedural manner is resuspended in hydrochloric ethanol (ethanol 80% by volume, adjusted with hydrochloric acid to pH 3.0) and is subsequently centrifuged. This washing out process is continued on the centrifuge by way of thrice repetition, wherein pyrrolizidine alkaloid residual values of maximal 1 ppm were ascertained in the thereby arisen pyrrolizidine-alkaloid-purified, second precipitate N2 (LC-MS/MS).

The filtrated second extract phase B as a mobile phase is now chromatographed in a chromatography column, which as a stationary phase is filled with an acidic cation exchanger. The eluate includes residual pyrrolizidine alkaloids of maximal 0.1 ppm (LC-MS/MS). The acidic catalyser based on polymer which is described above serves as a stationary phase. The eluate is set with caustic soda to pH 6.2, is concentrated by vacuum distillation and ethanol (96% by volume) is added. The pyrrolizidine-alkaloid-purified, second precipitate N2 is suspended and dissolved in the thus stabilised and purified second extract phase B and is subsequently supplemented with purified water.

The aforementioned method procedure results in a pyrrolizidine-alkaloid-purified special extract SE, also called special fluid extract, from symphytum, with a drug/extract ratio (DER) of 1:4.

1. A method for manufacturing a special extract (SE) from symphytum, wherein the method comprises the following steps:
providing a suspension system (SS) from symphytum,
differential precipitation of mucopolysaccharides (MPS), wherein the differential precipitation comprises the following steps:
first differential mucilage precipitation of high-molecular mucopolysaccharides (MPS) as a first precipitate (N1) in the suspension system (SS), separating the first precipitate (N1) and an extraction residue as a raffinate (R) and obtaining a first extract phase (A);
second differential mucilage precipitation of low-molecular mucopolysaccharides (MPS) as a second precipitate (N2) from the first extract phase (A), separating the second precipitate (N2) and obtaining a second extract phase (B);
selective removal of pyrrolizidine alkaloids (PA) from the second precipitate (N2) and/or the second extract phase (B); and
pooling the second precipitate (N2), in particular the second precipitate (N2) which is purified of the pyrrolizidine alkaloids, and the second extract phase (B), in particular the second extract phase (B) which is purified of the pyrrolizidine alkaloids, into the special extract (SE).

2. The method according to claim 1, wherein the pH-value of the suspension system is adjusted to 4-5 for the first differential mucilage precipitation.

3. The method according to claim 1, wherein the pH-value of the first extract phase (A) is adjusted to 2.5-3.5 for the second differential mucilage precipitation of low-molecular mucopolysaccharides (MPS)

4. The method according to claim 2, wherein the pH-value of the suspension system and/or of the first extract phase (A) is adjusted with the help of a cation exchanger in acidic form, wherein the cation exchanger is designed on the basis of an organic polymer resin and wherein the cation exchanger in particular comprises sulphonic acid groups on the polymer surface.

5. The method according to claim 1, wherein the special extract (SE) has a pyrrolizidine alkaloid content of maximally 0.5 ppm, in particular maximally 0.1 ppm.

6. The method according to claim 1, wherein the suspension system (SS) from symphytum can be manufactured by way of discontinuous extraction comprising the following steps:
providing a suspended, first, mucilage-free extraction phase (1) from symphytum; and
manufacturing a second, mucilaginous extraction phase (2).

7. The method according to claim 6, wherein the suspended, first extraction phase (1) can be provided by the following steps:
providing plant parts of symphytum;
adding low-molecular alcohol, in particular ethanol, to the plant parts;
setting the alcohol content of the first extraction phase (1) to 65-75% by volume, in particular to 70% by volume.

8. The method according to claim 7, wherein the provided plant parts of symphytum, to which low-molecular alcohol is added, are suspended by way of wet-comminution and/or turbo-extraction.

9. The method according to claim 6, wherein the second extraction phase (2) is manufactured by way of maceration, in particular stirring maceration, of the first extraction phase (1), wherein the alcohol content of the second extraction phase (2) is set to 45% by volume to 55% by volume, in particular to 50% by volume.

10. The method according to claim 1, wherein the selective removal of pyrrolizidine alkaloids (PA) from the second precipitate (N2) comprises the following steps:
resuspending the second precipitate (N2) in a washing solution, in particular in a washing solution comprising an acidic, low-molecular alcohol, wherein the alcohol in particular has a pH-value of 2-4, and
separating the washed, second precipitate (N2) from the washing solution. or
resuspending the second precipitate (N2) in high-percentage, low-molecular alcohol, in particular ethanol,
adding a catalyser, in particular an acidic catalyser based on a gel-like polymer, to the resuspended second precipitate (N2), wherein the catalyser in particular comprises sulphonic acid groups on the polymer surface, and
separating the catalyser from the resuspended, second precipitate (N2) and the subsequent recovery of the purified, second precipitate (N2) from the purified suspension;
wherein the resuspending and pyrrolizidine-alkaloid-purification can be repeated with the purified, second precipitate (N2); and
wherein the purified, second precipitate (N2) comprises in particular maximally 1 ppm pyrrolizidine alkaloids (PA).

11. The method according to claim 1, wherein the selective removal of pyrrolizidine alkaloids (PA) from the second extract phase (B) comprises at least one of the following steps:
pyrrolizidine-alkaloid-purification of the second extract phase (B) with the help of a stationary phase, wherein the stationary phase in particular comprises a cation exchanger in acidic form;
adjusting the pH-value of the pyrrolizidine-alkaloid-purified second extract phase (B) to 5.5-7, in particular to 6.2-6.5; and
concentrating the pyrrolizidine-alkaloid-purified second extract phase (B), in particular by way of distillation,
wherein the pyrrolizidine-alkaloid-purified and possibly concentrated second extract phase (B) comprises maximally 0.5 ppm pyrrolizidine alkaloids (PA), in particular maximally 0.25 ppm pyrrolizidine alkaloids (PA), in particular maximally 0.1 ppm pyrrolizidine alkaloids (PA).

12. The method according to claim 1, wherein at least one of the steps:
providing a suspension system (SS) from symphytum;
first differential mucilage precipitation of high-molecular mucopolysaccharides (MPS);
second differential mucilage precipitation of low-molecular mucopolysaccharides (MPS);
selective removal of pyrrolizidine alkaloids (PA) from the second precipitate (N2) and/or from the second extract phase (B)
is carried out under inert gas.

13. A special extract, manufacturable according to claim 1.

14 The special extract, manufacturable according to claim 1, for use in therapy.

15. The special extract, manufacturable according to claim 1 for use in the therapy of pain, inflammation, swelling, movement restrictions, bruises, strains, sprains, joint arthrosis, back pain, muscle ailments and/or joint ailments.

16. A method for manufacturing a suspended, first extraction phase (1) from symphytum comprising the following steps:
providing fresh, frozen and/or dried plant parts of symphytum;
adding low-molecular alcohol, in particular ethanol, to the plant parts;
setting the alcohol content of the first extraction phase (1) to 65-75% by volume, in particular to 70% by volume.

17. A method for manufacturing a second, mucilaginous extraction phase (2) from a provided first, essentially mucilage-free extraction phase (1) from symphytum, wherein the second, mucilaginous extraction phase (2) is manufactured by way of maceration, in particular by way of stirring maceration, of the first extraction phase (1), wherein the alcohol content of the extraction phase is set to 45% by volume-55% by volume, in particular to 50% by volume.

18. A method for precipitating mucopolysaccharides (MPS), comprising a differential mucilage precipitation, wherein the differential mucilage precipitation comprises the following steps:
first differential mucilage precipitation of high-molecular mucopolysaccharides (MPS) as a first precipitate (N1) in a suspension system (SS) from symphytum, separating the first precipitate (N1) and an extraction residue as a raffinate (R) and obtaining a first extract phase (A);
second differential mucilage precipitation of low-molecular mucopolysaccharides (MPS) as a second precipitate (N2) from the first extract phase (A), separating the second precipitate (N2) and obtaining a second extract phase (B).

19. A method for the selective removal of pyrrolizidine alkaloids (PA) from a precipitate which is precipitated from a suspension system (SS) from symphytum and is separated from an extract, comprising the following steps:
resuspending the precipitated and separated precipitate in a washing solution, in particular in a washing solution comprising an acidic, low-molecular alcohol, wherein the alcohol in particular has a pH-value of 2-4, and
separating the washed precipitate from the washing solution; or
resuspending the precipitated and separated precipitate in high-percentage, low-molecular alcohol, in particular ethanol,
adding a catalyser, in particular an acidic catalyser on the basis of a gel-like polymer, to the resuspended precipitate, wherein the catalyser in particular comprises sulphonic acid groups on the polymer surface, and
separating the catalyser from the resuspended precipitate and subsequent recovery of the purified precipitate from the suspension;
wherein the resuspending and purifying with the purified precipitate is repeatable; and
wherein the purified precipitate in particular comprises maximally 1 ppm of pyrrolizidine alkaloids (PA).

20. A method for the selective removal of pyrrolizidine alkaloids (PA) from an extract phase which is separated from a precipitate in a suspension system (SS) from symphytum, comprising at least one of the following steps:
purifying the extract phase with the help of a stationary phase, wherein the stationary phase in particular comprises a cation exchanger in acidic form;
adjusting the pH-value of the purified extract phase to 5.5-7, in particular to 6.2-6.5; and
concentrating the purified extract phase, in particular by way of distillation,
wherein the purified extract phase (B) comprises maximally 0.5 ppm of pyrrolizidine alkaloids (PA), in particular maximally 0.1 ppm of pyrrolizidine alkaloids (PA).
